(19) United States
(12) Patent Application Publication
(10) Pub. No.: US 2006/0074265 A1
(43) Pub. Date: Apr. 6, 2006

(54) Asymmetric catalytic systems

(76) Inventors: Julian Vaughan-Spickers, North Baddesley (GB); Eric George Hope, Harborough Magna (GB); Alison Stuart, East Goscote (GB); Andrew West, Cosby (GB)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/234,384

(22) Filed: Sep. 26, 2005

(30) Foreign Application Priority Data
Sep. 24, 2004 (EP) 04022878.05

(51) Int. Cl. C07C 39/12

(52) U.S. Cl. 568/719

(57) ABSTRACT

The present invention relates to a process for the recovery and the reuse of catalytic systems involving 1,1′-Bi-2-Naphthol (BINOL) and titanium tetraisopropoxide. A further object of the invention is a new method for catalyzing an asymmetric reaction in high conversion rate and enantioselectivity.

### TECHNICAL FIELD

The present invention relates to a process for the recovery and the reuse of catalytic systems involving 1,1′-Bi-2-Naphthol (BINOL) and titanium tetraisopropoxide. A further object of the invention is a new method for catalyzing an asymmetric reaction in high conversion rate and enantioselectivity.

### BACKGROUND ART

Asymmetric catalytic systems involving 1,1′-Bi-2-Naphthol (BINOL) and titanium tetraisopropoxide are well documented.

For example, Kitamoto et al. [Tetrahedron Lett., 36, 1995, 1861] described a chiral 1,1′-bi-2-naphthol(BINOL)-derived titanium complex, prepared via complete hydrolysis of the complex formed in situ by mixing (i-PrO)₄Ti and (R)—BINOL followed by azeotrope, which serves as a moisture-tolerable enantioselective catalyst for the glyoxylate-ene reaction and shows an asymmetric amplification therein.

In another reaction Imma et al. [Synlett, 12, 1996, 1229] used for an asymmetric catalytic hydrosilylation of ketones with HSi(OET)₃ (R)—BINOL—Ti(O—I—Pr)₂ as a precatalyst and observed levels of enantioselectivity up to 55 % and an “enantioselective autoinduction” of the reaction.

Zhang et al. [ Tetrahedron Asymmetry, 8, 1997, 585] described an enantioselective addition of diethylzinc to aldehydes by using a catalyst prepared in situ by mixing titanium tetraisopropoxide with S— or R—BINOL.

The asymmetric catalytic alkylation of aldehydes with diethylzinc using (R)—BINOL—Ti(O—I—Pr)₂ complex as an asymmetric precatalyst is disclosed by Mori and Nakai [Tetrahedron Lett., 38, 1997, 6233] in order to afford the corresponding secondary alcohols.

However, all these systems are interested solely in the abilities of the catalyst and not in the recovery and reuse of the ligand/catalyst.

Despite the relatively short period these BINOL/Ti systems have been studied, a number of groups have worked on recovering the ligand/catalyst. These approaches have included the use of fluorous biphasic conditions [Tian, Y., Yang, Q. C., Mak, T. C. W., Chan, K. S., Tetrahedron, 58, 2002, 3951; Nakamura, Y., Takeuchi, S., Ohgo, Y., Curran, D. P., Tetrahedron Lett., 41, 2000, 57]. The main problems with all of the attempted recycling systems are that they require extensive functionalisation of the binaphthyl backbone before separation is achievable and, in many cases, the activity and selectivity of the modified BINOL catalysts are reduced in comparison to the unmodified systems, making the techniques employed for catalyst recovery detrimental to the overall catalytic process.

### AIMS OF THE INVENTION

In view of the above, and due to the expensive nature of the resolved BINOL, ligand recovery is an aim of the present invention. It is therefore an object of the present invention to present a process for the separation and recovery of a BINOL ligand/catalyst as an alternative to the methods of recovery which have failed. A further object of the invention is to provide both a new method for catalyzing an asymmetric reaction in high conversion rate and enantioselectivity and for recovering and re-using of the ligand/catalyst without falls in activity or specificity.

### DISCLOSURE OF THE INVENTION

The present invention relates to a light fluorous approach to improving the method for the recovery and recycling of the synthetically important BINOL/Ti catalytic system. The term “light fluorous approach” is well understood in the art to loosely refer to perfluorinated tagging molecules, where the perflurorinated part is a minor portion of the hydrocarbon of the compound. A discussion of the light fluorous approach can be found in the “Handbook of Fluorous Chemistry,” Wiley (2004), Gladysz, J. A. ; Curran, D. P. Hvath , I. T., which is incorporated herein by reference for its teachings on the light fluorous approach.

As a result of experiments a methodology is developed for the allylation of aldehydes. It has been found that tagging the BINOL moiety with fluorinated side-chains and using a solid support, for example, a fluorous reverse phase silica, enables the separation of products from catalyst.

The invention in further aspects relates to a process for the preparation of asymmetrically substituted compounds using a light fluorous approach, in which a fluoroalkyl tagged 1,1′-Bi-2-Naphthol [(S)— or (R)—BiNOL] derivative and titanium tetraisopropoxide catalytic system is used,characterized in that the catalytic system is recovered after the reaction by

a) adding the crude mixture on the top of a reverse phase chromatography column

b) eluting the reaction product,

c) isolating the formed Sn-Rf-BINOL polymeric material, which is formed during the asymmetric catalytic reaction,

d) recovering free (S)— or (R)-Rf-BINOL from the solution received in step c) with an acidified non-polar solvent and

e) reusing the recovered (S)— or (R)-Rf-BINOL in an asymmetric catalytic reaction.

The crude mixture typically comes from a reaction vessel where the catalytic reaction has occurred, typically followed by conventional work-up prior to transfer to the top of the chromatography column. The crude mixture, e.g., an oil resulting from the work-up, contains the Sn-Rf-BINOL polymeric material and any unreacted (S)— or (R)-Rf-BINOL. The titanium tetraisopropoxide component of the catalytic system is typically lost in the work-up and is newly added again in subsequent reaction runs.

The recovery of free (S)— or (R)-Rf-BINOL from the solution containing the Sn-Rf-BINOL polymeric material with an acidified non-polar solvent is believed to involve an acid-hydrolysis reaction.

Asymmetrically substituted reactions, including the reactants used therein and the products of the reactions, are well known in the art. Examples of the type of reactions included can be found in “Catalytic Asymmetric Synthesis,” Wiley (2000), Iwao, O., which is incorporated herein by reference for its teaching on asymmetrically substituted reactions. Asymmetric reactions include, for example, Diels-Alder, Cycloaddition, Mannich, Michael and Aldol.

This process in further aspects is characterized in that the chromatography column is packed with a suitable solid support selected from the group silica gel, FRP silica gel, C₈ reverse phase silica gel, and powdered poly(tetrafluoroethene); or in that the chromatography column is packed with a fluorous reverse phase silica gel; or in that the formed Sn-Rf-BINOL polymeric material is isolated from the solid support by the use of an aprotic polar solvent; or in that the formed Sn-Rf-BINOL polymeric material is isolated from the solid support by the use of diethyl ether; or in that in washing off the non-polar Sn-Rf-BINOL polymeric material and any free (S)— or (R)-Rf-BINOL with a fluorophilic solvent selected from the group diethyl ether, tetrahydrofuran, acetone and perfluorinated solvents; or in that for recovering free (S)— or (R)-Rf-BINOL from the solution received in step c) a non-polar solvent is acidified with a weakly acidic solution; or in that for recovering free (S)— or (R)-Rf-BINOL from the solution received in step c) a non-polar solvent, such as hexane, benzene, toluene, xylene, pentane, etc., preferably, hexane, is acidified with a weakly acidic solution obtained from, for example, the mineral acid hydrochloric acid or sulphuric acid or nitric acid, etc., preferably, hydrochloric acid; or in that it is useful for the production of asymmetric materials.

The present invention further includes a method to regenerate the catalytic system from a new Sn(Bu)₃-BINOL polymer.

A further object of the present invention is to illustrate that this system does not decrease catalytic activity/efficiency.

In summary, the present invention provides a route to enable the recycling of a BINOL/Ti catalyst and demonstrates that this can be isolated and reused without any decrease in activity.

While the description when describing certain embodiments focuses on (R)-Rf-BINOL, or (R)—BINOL, all reactions and methods described for any one BINOL system of this invention is equally applicable to other BINOL systems, for example, to (R)-Rf-BINOL, R—BINOL, (S)-Rf-BINOL and (S)—BINOL.

Comparison of the Two (R)—BINOL Species

A study was performed on the addition of allyltri-n-butyltin to benzaldehyde (allyltri-n-butyltin is a common reagent for the addition of allyl groups to organic substrates and is, therefore, cheap and easily obtained) using both (R)—BINOL an (R)-Rf-BINOL in order to ascertain whether conversions and enantiomeric excesses (ees) would be effected by the presence of perfluoroalkyl groups (Rf=C6F13). The reaction scheme is as follows:

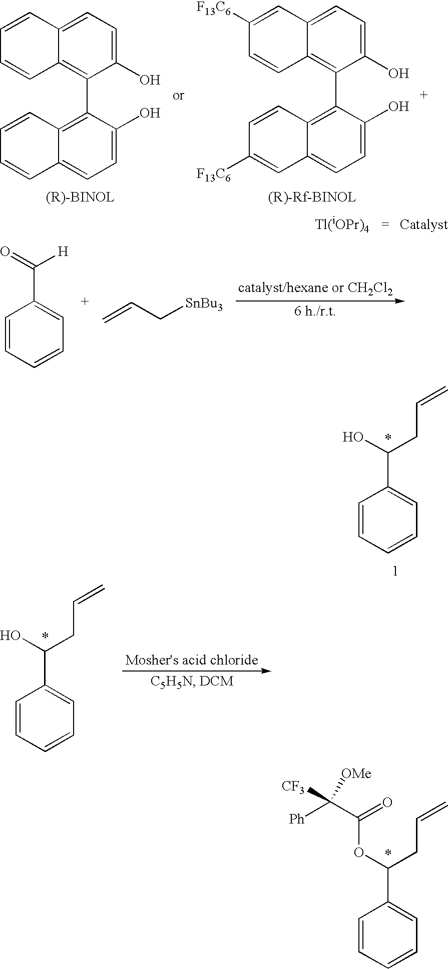

The progress of the reaction was determined by the conversion into the Mosher's acid ester followed by GC analysis.

The results after three separate reaction runs for (R)—BINOL and (R)-Rf-BINOL are summarized in the following table:

| Ligand | Conversion (%)^{a} | ee (%)^{b} |
|---|---|---|
| (R)-BINOL^{c} | 90 | 82 |
| (R)-BINOL^{c} | 90 | 78 |
| (R)-BINOL^{c} | 88 | 78 |
| (R)-Rf-BINOL^{d} | 86 | 74 |
| (R)-Rf-BINOL^{d} | 90 | 78 |
| (R)-Rf-BINOL^{d} | 88 | 74 |

^{a}By ¹H NMR
^{b}By GC of Mosher's acid ester
^{c}In DCM, O° C., t = 6 h
^{d}In hexane, O° C., t = 6 h

The results show that the inclusion of perfluoro-alkyl chains has no detrimental effect on the modified ligand. Also, the reaction is highly reproducible, with similar product conversions and enantiopurities observed after three separate runs.

Separation of the Ligand from the Product (4-phenyl-1-buten-4-ol 1)

After each catalytic run the reaction mixture was concentrated in vacuo. After concentration of a reaction using (R)—BINOL, crystals were noted to form. After NMR, MS and X-ray crystal analysis these were shown to be of a previously unreported Sn(Bu)₃-BINOL polymer. The tin-containing residue in this case, Su (Bu)₃, which can optionally contain various substituents in addition to or instead of or on one or more of the Bu groups, binds to the oxygens of BINOL forming a polymeric chain, and this is a very interesting structure because it suggests that the material to be isolated at the end of the reaction and reused may not be a titanium-containing species at all. Also, formation of this Sn—BINOL polymer clearly changes the separation dynamics of the mixture, suggesting that a non-polar solid separation media, such as fluorous reverse phase silica gel, should be much more appropriate for ligand retention.

Next, a standard catalytic addition was carried out using (R)-Rf-BINOL as the ligand. After concentration in vacuo the crude mixture was added to the top of a short column of fluorous reverse phase silica gel and eluted with acetonitrile. Solvent was removed from the isolated material to yield a yellow oil which was shown to be 4-phenyl-1-buten-4-ol 1 by ¹H NMR, and there was no evidence of (R)-Rf-BINOL. The product conversion was determined to be 89% by ¹H NMR analysis and the ee to be 70% by ¹H NMR analysis of the Mosher's acid ester of the product. After removing the Sn-Rf-BINOL polymeric material from the column using diethyl ether, it was attempted to use this material in a standard catalytic run. Unfortunately, there was no evidence of reaction and only starting material was observed. This indicates that the polymeric species is not catalytically active. It can be envisaged that retention of the material on the column of fluorous reverse phase silica gel is due to the moiety's highly hydrophobic nature and subsequent favourable non-polar Van der Waals interactions with the solid support. This allows the relatively polar acetonitrile solvent to wash off the product from the column while leaving behind the non-polar Sn-Rf-BINOL polymeric material and any free (R)-Rf-BINOL. Diethyl ether, which is a very fluorophilic solvent, can then be used to recover the Sn-Rf-BINOL polymeric material and it was shown that the free (R)-Rf-BINOL can be recovered by washing this species, dissolved in hexane, with 4M hydrochloric acid. The Sn-Rf-BINOL likewise to the Sn (Bu)₃-BINOL polymer, can contain 3 Bu groups, i.e., the Sn group can represent a tin containing residue Sn (Bu)₃, which is as defined previously.

Recovered (R)-Rf-BINOL was then used in three further catalytic runs using the same catalysis, separation and recovery methodology. The results are as follows:

| Run | Conversión (%)^{a} | ee (%)^{b} |
|---|---|---|
| 1 | 85 | 66 |
| 2^{c} | 85 | 63 |
| 3^{c} | 82 | 58 |
| 4^{c} | 78 | 58 |

^{a}By ¹H NMR
^{b}By GC of Mosher's acid ester
^{c}Using ligand from previous run

These data show that similar product conversions are achieved after each run, with a slight fall after the third ligand reuse. This is most likely due to mechanical losses of the ligand after each recycle rather than any deterioration of the ligand itself. The fall in product ee is also slight, and is probably due to a small amount of ligand racemisation. The conversions to product using this methodology are better than those observed previously and, unlike previously, the data shows the ligand can be successfully recycled after use in catalysis.

Several supports were investigated and results have indicated that fluorous reverse phase silica gel is a preferred support for separation and recovery of the (R)-Rf-BINOL ligand.

In a preferred embodiment of the invention, in the Rf-BINOL system the use of acetonitrile as eluant allows the ligand and product to be successfully separated using fluorous reverse phase silica gel. Then, with elution using diethyl ether followed by an acid wash, free (R)-Rf-BINOL can be recovered and reused in further catalysis without loss of activity or selectivity. Other polar solvents useful for eluting product include acetone, dimethylformamide and dimethyl sulfoxide.

In other embodiments, silica gel, C₈ reverse phase silica gel or powdered Teflon are used as the solid support, however, leaching of the ligand-based species and contamination of the product may occur.

Preferably, Rf groups can be C4F9 to C12F25 and substitution of the BINOL can be at the 3, 4 and 6 positions.

In sum, a fluoroalkyl “R” tagged BINOL is any BINOL with one or more fluoroalkyl groups attached to one or both of the Naphthol groups of the BINOL. The Rf groups preferably are perfluorinated or highly fluorinated alkyl groups, e.g., more than 50% of substituents on the alkyl group are fluorine goups, e.g., more than about 60%, 70%, 80%, 90%, or 95%, and can be, for example, C₄F₉ to C₁₂F₂₅, more preferably C₆F₁₃. Additionally, the Rf groups attached to the naphthol group(s) of the BINOL can optionally and independently of each other contain a —CH₂— or —CH₂—CH₂— group between the fluorinated or highly fluorinated part of the Rf group. Thus, preferred groups for Rf include, e.g., —CH₂—C₄F₉ to —CH₂—C₁₂F₂₅ and —CH₂—CH₂—C₄F₉ to —CH₂—CH₂—C₁₂F₂₅, and more preferably —CH₂—C₆F₁₃ and —CH₂—CH₂—C₆F₁₃. The Rf group may be a straight chained or branched chain fluoralkyl group. The locations of the Rf groups is preferably in the 3, 4 and/or 6 positions, and preference is given to embodiments where each of the Naphthol groups have Rf groups in corresponding symmetric positions, i.e., in the 3, 3′, 4, 4′, 6, and/or 6′ positions.

In sum, (R)-Rf-BINOL is used in an asymmetric catalytic reaction in combination with a titanium tetraisopropoxide catalytic system. The (R)-Rf-BINOL during the reaction is converted to a Sn-Rf-BINOL polymeric material, which along with some un-reacted (R)-RF-BINOL, if any, is retained on the solid support of the column. The product is eluted from the support by washing with a polar solvent, for example, a polar acetonitrile solvent, leaving behind the polar Sn-Rf-BINOL polymeric material and any unreacted (R)-Rf-BINOL on the column. The Sn-Rf-BINOL polymeric material and any (R)-Rf-BINOL is removed from the support by washing the same with an aprotic polar solvent, for example, a fluorophilic solvent, for example, diethyl ether, tetrahydrofuran, acetone or other perfluorinated solvents. The Sn-Rf-BINOL polymeric material removed from the support is not catalytically active, and prior to reuse in another reaction, is converted to (R)-Rf-BINOL. This conversion can be achieved by using an acidified non-polar solvent, for example, hexane, which is acidified, for example, with a weakly acidic solution, for example, with a solution obtained from the mineral acid hydrochloric acid.

The scope of this methology is wide and in principle can be applied to any system using BINOL (e.g. alkylation of benzaldehyde using diethylzinc). In general any Lewis acid-catalysed reactions will work.

Addition of the allyl group and subsequent separation is performed as described above.

In preferred aspects the invention relates to:

A process for regenerating (R)-Rf-BINOL or (S)-Rf-BINOL in which Rf signifies that one or more fluoroalkyl groups are substituting R-(+)-1,1′-Bi-2-Naphthol or S-(−)-1,1′-Bi-2-Naphthol, comprising reacting an Sn-Rf-BINOL polymeric material, Sn standing for tin or a tin-containing group, with an acidified non-polar solvent to obtain (R)-Rf-BINOL or (S)-Rf-BINOL; and

In a process for preparing an asymmetrically substituted compound using a light fluorous approach with a catalyst containing
(R)-Rf-BINOL or (S)-Rf-BINOL, in which Rf signifies that one or more fluoroalkyl groups are substituting R-(+)-1,1′-Bi-2-Naphthol or S-(−)-1,1′-Bi-2-Naphthol,
and
a titanium tetraisopropoxide compound,

the improvement comprising using (R)-Rf-BINOL or (S)-Rf-BINOL in the catalyst that has been regenerated by a process for regenerating (R)-Rf-BINOL or (S)-Rf-BINOL as described above; and

A process for regenerating (R)-Rf-BINOL or (S)-Rf-BINOL as described above, wherein the non-polar solvent is hexane, benzene, toluene, xylene, or pentane; and

A process for regenerating (R)-Rf-BINOL or (S)-Rf-BINOL as described above, wherein the acidified non-polar solvent is acidified with a weakly acidic solution; and

A process for regenerating (R)-Rf-BINOL or (S)-Rf-BINOL as described above, wherein the acidified non-polar solvent is acidified with a weakly acidic solution obtained from the mineral acid hydrochloric acid or sulphuric or nitric acid; and

A process for regenerating (R)-Rf-BINOL or (S)-Rf-BINOL as described above, wherein Rf is —L—C₄F₉ to —L—C₁₂F₂₅, wherein L is a direct bond, —CH₂— or —CH₂—CH₂—; and

A process for regenerating (R)-Rf-BINOL or (S)-Rf-BINOL as described above, wherein Rf is —L—C₆F₁₃, whereinL is a direct bond, —CH₂— or —CH₂—CH₂—; and

A process for regenerating (R)-Rf-BINOL or (S)-Rf-BINOL as described above, wherein Rf group or groups are present at the 3, 4 and/or 6 positions of the R-(+)-1,1′-Bi-2-Naphthol or S-(−)-1,1′-Bi-2-Naphthol compound on either one or both Naphthol groups; and

A process for regenerating (R)-Rf-BINOL or (S)-Rf-BINOL as described above, wherein the (R)-Rf-BINOL is

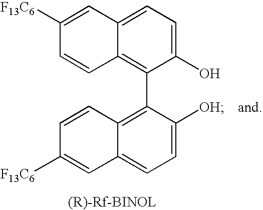

A process for regenerating (R)-Rf-BINOL or (S)-Rf-BINOL as described above, wherein Sn is a tin containing group wherein the tin is bonded to 3 butyl groups; and

A process for regenerating (R)-Rf-BINOL or (S)-Rf-BINOL as described above, wherein (R)-Rf-BINOL is regenerated.

In further preferred aspects the invention relates to:

A process for recovering (R)-Rf-BINOL or (S)-Rf-BINOL for use in a catalyst, in which

the catalyst contains
(R)-Rf-BINOL or (S)-Rf-BINOL, in which Rf signifies that one or more fluoroalkyl groups are substituting R-(+)-1,1′-Bi-2-Naphthol or S-(−)-1,1′-Bi-2-Naphthol,
and
a titanium tetraisopropoxide compound,

the process comprising:

a) reacting and then passing a mixture through a reverse phase chromatography column packed with a solid support during which reaction the (R)-Rf-BINOL or (S)-Rf-BINOL becomes an Sn-Rf-BINOL polymeric material that binds to the solid support, Sn standing for tin or a tin-containing group,

b) removing the Sn-Rf-BINOL polymeric material from the solid support, and

c) reacting the removed Sn-Rf-BINOL polymeric material with an acidified non-polar solvent to obtain (R)-Rf-BINOL or (S)-Rf-BINOL; and

A process for recovering (R)-Rf-BINOL or (S)-Rf-BINOL for use in a catalyst as described above, further comprising using the (R)-Rf-BINOL or (S)-Rf-BINOL obtained from the Sn-Rf-BINOL polymeric material in a process for preparing an asymmetrically substituted compound; and

A process for recovering (R)-Rf-BINOL or (S)-Rf-BINOL for use in a catalyst as described above, wherein the solid support is silica gel, FRP silica gel, C₈ reverse phase silica gel, or powdered poly(tetrafluoroethene); and

A process for recovering (R)-Rf-BINOL or (S)-Rf-BINOL for use in a catalyst as described above, wherein the solid support is a fluorous reverse phase silica gel; and

A process for recovering (R)-Rf-BINOL or (S)-Rf-BINOL for use in a catalyst as described above, wherein the Sn-Rf-BINOL polymeric material is removed from the solid support by an aprotic polar solvent, by a fluorophilic solvent, by diethyl ether, tetrahydrofuran, acetone or a perfluorinated solvent; and

A Sn-Rf-BINOL polymeric material formed by a process for recovering (R)-Rf-BINOL or (S)-Rf-BINOL for use in a catalyst as described above.

In still further preferred aspects the invention relates to:

In a process for preparing an asymmetrically substituted compound using a light fluorous approach with a catalyst containing
(R)-Rf-BINOL or (S)-Rf-BINOL, in which Rf signifies that one or more fluoroalkyl groups are substituting R-(+)-1,1′-Bi-2-Naphthol or S-(−)-1,1 ′-Bi-2-Naphthol,
and
a titanium tetraisopropoxide compound,

the improvement comprising using (R)-Rf-BINOL or (S)-Rf-BINOL in the catalyst that has been regenerated by a process comprising

a) reacting and then passing a mixture through a reverse phase chromatography column packed with a solid support during which reaction the (R)-Rf-BINOL or (S)-Rf-BINOL becomes an Sn-Rf-BINOL polymeric material that binds to the solid support, Sn standing for tin or a tin-containing group,

b) removing the Sn-Rf-BINOL polymeric material from the solid support,

c) reacting the removed Sn-Rf-BINOL polymeric material with an acidified non-polar solvent to obtain (R)-Rf-BINOL or (S)-Rf-BINOL, and

d) eluting from the solid support with a polar solvent a product that is an asymmetrically substituted compound which is a product of the reaction of the mixture in a); and

In a process for preparing an asymmetrically substituted compound using a light fluorous approach as described above, wherein the polar solvent is an acetonitrile solvent, acetone, dimethylformamide or dimethyl sulfoxide.

In further preferred aspects the invention relates to:

A process for recovering (R)—BINOL or (S)—BINOL for use in a catalyst, in which

the catalyst contains
(R)—BINOL, which is R-(+)-1,1′-Bi-2-Naphthol, or (S)—BINOL, which is S-(−)-1,1′-Bi-2-Naphthol,
and
a titanium tetraisopropoxide compound,

wherein the (R)—BINOL or (S)—BINOL is recovered from a Sn(Bu)₃-BINOL polymeric material formed during a reaction in the process, Sn standing for tin, and Bu standing for butyl,

the process comprising:

a) reacting a mixture in a reverse phase chromatography column packed with a solid support during which reaction the (R)—BINOL or (S)—BINOL becomes an Sn(Bu)₃-BINOL polymeric material that binds to the solid support,

b) removing the Sn(Bu)₃-BINOL polymeric material from the solid support,

c) reacting the removed Sn(Bu)₃-BINOL polymeric material with an acidified non-polar solvent to obtain (R)—BINOL or (S)—BINOL; and

A Sn(Bu)₃-BINOL polymeric material formed by a process for recovering (R)—BINOL or (S)—BINOL for use in a catalyst as described above.

Additionally, in further preferred aspects the invention relates to:

The (R)-Rf-BINOL and (S)-Rf-BINOL compounds themselves where these compounds, including the Rf groups are as defined above.

### INDUSTRIAL APPLICABILITY

The light fluorous approach of the present invention enables the expensive, industrially useful, BINOL/Ti asymmetric catalytic system to be recovered and recycled. This will enable the costs of many synthetically common reactions to be reduced.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

### EXAMPLES

General Catalysis Procedure for the Addition of Allyltri-n-butyltin to Benzaldehyde

To a solution of Ti(OⁱPr)₄ (0.3 ml in 20 ml hexane, 2 ml, 0.1 mmol), is added the BINOL ligand (0.2 mmol) and the mixture is stirred for one hour. Then the mixture is cooled to 0° C. and benzaldehyde (0.1 ml, 1 mmol) is added. The mix is stirred for 10 minutes and then allyltri-n-butyltin is added (0.34 ml, 1.1 mmol) and the reaction mixture is held at 0° C. for six hours.

The reaction is quenched with saturated NaHCO₃ solution (5 ml), washed with 1M hydrochloric acid (10 ml) and the mixture is filtered, dried (MgSO₄), filtered once more and the solvent removed in vacuo to yield the product as a colourless oil contaminated with ligand.

General Procedure for the Separation of BINOL and 4-phenyl-1-buten-4-ol

A standard reaction mixture is concentrated in vacuo and the residue is placed onto the top of a column of silica gel, FRP silica gel, C₈-reverse phase silica gel or powdered PTFE 3 cm long and 1 cm in diameter. Acetonitrile is then used as elutant to recover the product. Diethyl ether is used as the second elutant to recover any ligand not eluted with the acetonitrile phase. When FRP silica gel and (R)-Rf-BINOL are used complete separation of the ligand and product are achieved. (R)-Rf-BINOL is used in three further catalytic runs following the same catalysis procedure using the recovered ligand. The general separation procedure is then used to recover the ligand and separate the 4-phenyl-1-buten-4-ol product. After each run, the product is washed with 6M hydrochloric acid and the Sn and Ti levels are determined by ICP analysis of the wash. Figures in brackets indicate percentage of metal added at the outset that is present in the product. After separation of the ligand on FRP silica gel, the ether fractions are combined, washed with 4M hydrochloric acid, dried with MgSO₄ and the solvent is removed to yield (R)-Rf-BINOL. Run 1: 85% conversion, 66% ee. Sn: 1.24 ppm, (0.02%) Ti: 2.21 ppm, (0.88%); Run 2: 85% conversion, 63% ee. Sn: 0.75 ppm, (0.01%) Ti: 1.99 ppm, (0.83%); Run 3: 82% conversion, 58% ee. Sn: 2.73 ppm, (0.04%) Ti: 2.86 ppm, (1.19%); Run 4: 78% conversion, 58% ee. Sn: 4.40 ppm, (0.07%) Ti: 4.65 ppm, (1.93%).

Asymmetric Addition of Allyltri-n-butyltin Using (R)—BINOL

The general catalysis procedure is followed using (R)—BINOL (57 mg, 0.2 mmol) as ligand. The product is collected as a colourless oil contaminated with ligand. m/z (ES-) 149 [MH]⁻ (23%). δ_{H} 2.32 (1H, bs, OH), 2.54 (2H, um, CH₂), 4.74 (1H, dd, ³J_{HH} 5.8 Hz, ³J_{HH} 7.0 Hz, CH), 5.15 (1H, um, ═CH₂), 5.20 (1H, dum, ³J_{HH} 9.0 Hz, ═CH₂), 5.84 (1H, um, HC═), 7.24-7.39 (5H, um, PhH).

Asymmetric Addition of Allyltri-n-butyltin Using (R)-Rf-BINOL

The general catalysis procedure is followed using (R)-Rf-BINOL (184 mg, 0.2 mmol) as ligand. The product is collected as a colourless oil. m/z (ES-) 149 [MH]⁻ (26%). δ_{H} 2.32 (1H, bs, OH), 2.54 (2H, um, CH₂), 4.74 (1H, dd, ³J_{HH} 5.8 Hz, ³J_{HH} 7.0 Hz, CH), 5.15 (1H, um, ═CH₂), 5.20 (1H, dum, ³J_{HH} 9.0 Hz, ═CH₂), 5.84 (1H, um, HC═), 7.24-7.39 (5H, um, PhH).

Procedure for the Determination of 4-Phenyl-1-buten-4-ol Product ee

Mosher's acid chloride (250 mg in 10 ml DCM, 990 μM, 2 ml, 0.2 mmol) and pyridine (0.2 ml, 2.5 mmol) are added to a flame-dried flask under nitrogen.

To this mixture, 4-phenyl-1-buten-4-ol (29 μl, 0.2 mmol) is added and the reaction mixture is stirred for 30 minutes.

1M hydrochloric acid is added (3 ml) and the biphase is transferred to a separating funnel. The organic phase is separated and washed with NaHCO₃ (10 ml) and water (10 ml), dried (MgSO₄), filtered and the solvent removed in vacuo to yield a colourless oil which is analysed by chiral GC for diastereomeric content (CYDEX—B, 180° C. for 20 min. Injector: 220° C., detector: 250° C. Flow rate: 2 ml/min (R)-4-phenyl-1-buten-4-ol, Mosher's acid ester Rₜ 8.81 min, (S)-4-phenyl-1-buten-4-ol, Mosher's acid ester, Rₜ 13.00 min).

The entire disclosure[s] of all applications, patents and publications, cited herein and of corresponding EP application No. 04022878.5, filed Sep. 24, 2004, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A process for regenerating (R)-Rf-BINOL or (S)-Rf-BINOL in which Rf signifies that one or more fluoroalkyl groups are substituting R-(+)-1,1′-Bi-2-Naphthol or S-(−)-1,1′-Bi-2-Naphthol, comprising reacting an Sn-Rf-BINOL polymeric material, Sn standing for tin or a tin-containing group, with an acidified non-polar solvent to obtain (R)-Rf-BINOL or (S)-Rf-BINOL.

2. In a process for preparing an asymmetrically substituted compound using a light fluorous approach with a catalyst containing
(R)-Rf-BINOL or (S)-Rf-BINOL, in which Rf signifies that one or more fluoroalkyl groups are substituting R-(+)-1,1′-Bi-2-Naphthol or S-(−)-1,1 ′-Bi-2-Naphthol,
and
a titanium tetraisopropoxide compound,
the improvement comprising using (R)-Rf-BINOL or (S)-Rf-BINOL in the catalyst that has been regenerated by a process according to claim 1.

3. A process according to claim 1, wherein the non-polar solvent is hexane, benzene, toluene, xylene, or pentane.

4. A process according to claim 1, wherein the acidified non-polar solvent is acidified with a weakly acidic solution.

5. A process according to claim 1, wherein the acidified non-polar solvent is acidified with a weakly acidic solution obtained from the mineral acid hydrochloric acid or sulphuric or nitric acid.

6. A process according to claim 1, wherein Rf is —L—C₄F₉ to —L—C₁₂F₂₅, wherein L is a direct bond, —CH₂— or —CH₂—CH₂—.

7. A process according to claim 1, wherein Rf is —L—C₆F₁₃, whereinL is a direct bond, —CH₂— or —CH₂—CH₂—.

8. A process according to claim 1, wherein Rf group or groups are present at the 3, 4 and/or 6 positions of the R-(+)-1,1′-Bi-2-Naphthol or S-(−)-1,1′-Bi-2-Naphthol compound on either one or both Naphthol groups.

9. A process according to claim 1, wherein the (R)-Rf-BINOL is

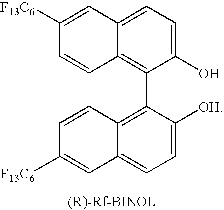

10. A process according to claim 1, wherein Sn is a tin containing group wherein the tin is bonded to 3 butyl groups.

11. A process for recovering (R)-Rf-BINOL or (S)-Rf-BINOL for use in a catalyst, in which
the catalyst contains
(R)-Rf-BINOL or (S)-Rf-BINOL, in which Rf signifies that one or more fluoroalkyl groups are substituting R-(+)-1,1 ′-Bi-2-Naphthol or S-(−)-1,1′-Bi-2-Naphthol,
and
a titanium tetraisopropoxide compound,
the process comprising:
a) reacting and then passing a mixture through a reverse phase chromatography column packed with a solid support during which reaction the (R)-Rf-BINOL or (S)-Rf-BINOL becomes an Sn-Rf-BINOL polymeric material that binds to the solid support, Sn standing for tin or a tin-containing group,
b) removing the Sn-Rf-BINOL polymeric material from the solid support, and
c) reacting the removed Sn-Rf-BINOL polymeric material with an acidified non-polar solvent to obtain (R)-Rf-BINOL or (S)-Rf-BINOL.

12. A process according to claim 11, further comprising using the (R)-Rf-BINOL or (S)-Rf-BINOL obtained from the Sn-Rf-BINOL polymeric material in a process for preparing an asymmetrically substituted compound.

13. A process according to claim 11, wherein the solid support is silica gel, FRP silica gel, C₈ reverse phase silica gel, or powdered poly(tetrafluoroethene).

14. A process according to claim 11, wherein the solid support is a fluorous reverse phase silica gel.

15. A process according to claim 11, wherein the Sn-Rf-BINOL polymeric material is removed from the solid support by an aprotic polar solvent, by a fluorophilic solvent, by diethyl ether, tetrahydrofuran, acetone or a perfluorinated solvent.

16. In a process for preparing an asymmetrically substituted compound using a light fluorous approach with a catalyst containing
(R)-Rf-BINOL or (S)-Rf-BINOL, in which Rf signifies that one or more fluoroalkyl groups are substituting R-(+)-1,1′-Bi-2-Naphthol or S-(−)-1,1 ′-Bi-2-Naphthol,
and
a titanium tetraisopropoxide compound,
the improvement comprising using (R)-Rf-BINOL or (S)-Rf-BINOL in the catalyst that has been regenerated by a process comprising
a) reacting and then passing a mixture through a reverse phase chromatography column packed-with a solid support during which reaction the (R)-Rf-BINOL or (S)-Rf-BINOL becomes an Sn-Rf-BINOL polymeric material that binds to the solid support, Sn standing for tin or a tin-containing group,
b) removing the Sn-Rf-BINOL polymeric material from the solid support,
c) reacting the removed Sn-Rf-BINOL polymeric material with an acidified non-polar solvent to obtain (R)-Rf-BINOL or (S)-Rf-BINOL, and
d) eluting from the solid support with a polar solvent a product that is an asymmetrically substituted compound which is a product of the reaction of the mixture in a).

17. A process according to claim 16, wherein the polar solvent is an acetonitrile solvent, acetone, dimethylformamide or dimethyl sulfoxide.

18. A process for recovering (R)—BINOL or (S)—BINOL for use in a catalyst, in which
the catalyst contains
(R)—BINOL, which is R-(+)-1,1′-Bi-2-Naphthol, or (S)—BINOL, which is S-(−)-1,1′-Bi-2-Naphthol,
and
a titanium tetraisopropoxide compound,
wherein the (R)—BINOL or (S)—BINOL is recovered from a Sn(Bu)₃-BINOL polymeric material formed during a reaction in the process, Sn standing for tin, and Bu standing for butyl,
the process comprising:
a) reacting a mixture in a reverse phase chromatography column packed with a solid support during which reaction the (R)—BINOL or (S)—BINOL becomes an Sn(Bu)₃-BINOL polymeric material that binds to the solid support,
b) removing the Sn(Bu)₃-BINOL polymeric material from the solid support,
c) reacting the removed Sn(Bu)₃-BINOL polymeric material with an acidified non-polar solvent to obtain (R)—BINOL or (S)—BINOL.

19. A Sn(Bu)₃-BINOL polymeric material formed by a process according to claim 19.

20. A Sn-Rf-BINOL polymeric material formed by a process according to claim 11.

21. A process according to claim 1, wherein (R)-Rf-BINOL is regenerated.
